(19) United States
(12) Patent Application Publication
(10) Pub. No.: US 2024/0069025 A1
(43) Pub. Date: Feb. 29, 2024

(54) METHOD FOR SIZE BASED EVALUATION OF PANCREATIC PROTEIN MIXTURE

(71) Applicant: Kashiv BioSciences, LLC, Piscataway, NJ (US)

(72) Inventors: Raja Reddy Kuppili, Thane (IN); Roshan Ganeshial Upadhyay, Ahmedabad (IN); Parva Yogeshchandra Purohit, Ahmedabad (IN); Mukesh Mahajan, Ahmedabad (IN); Sanjaykumar Vanrajbhat Talpara, Ahmedabad (IN)

(21) Appl. No.: 18/262,318

(22) PCT Filed: Mar. 23, 2022
(86) PCT No.: PCT/IB2022/052652

§ 371 Date: Jul. 20, 2023

(30) Foreign Application Priority Data
Mar. 23, 2021 (IN) 202121012563

(51) Int. Cl. G01N 33/573; G01N 33/68

(52) U.S. Cl. CPC G01N 33/573; G01N 33/6851; G01N 2333/92; G01N 2333/928; G01N 2333/96433

(57) ABSTRACT

The present invention provides an improved method for analysis of pancreatic protein mixture comprises at least more than one biological active protein selected from amylase, protease and lipase, wherein the analysis and quantification of pancreatic protein mixtures is performed with Size Exclusion High Performance Liquid Chromatography (SE-HPLC). Also, the present invention provides the process for the separation, analysis and quantification of low molecular weight and high molecular weight enzymes present in pancreatic protein mixture.

### FIELD OF THE INVENTION

The presents invention provides an improved method for analysis of pancreatic protein mixture comprises at least more than one biological active protein selected from amylase, protease, lipase and mixture thereof, wherein the analysis and/or quantification of pancreatic protein mixtures is performed with Size Exclusion High Performance Liquid Chromatography (SE-HPLC).

### BACKGROUND OF THE INVENTION

Pancreatic enzymes produced by the body are well known for the integral role they play in the digestion of the foods. Pancreatic juice contains numerous enzymes, including amylase, lipase, protease, cholesterol esterase, and phospholipase, and the proenzymes trypsinogen, chymotrypsinogen, and procarboxypolypeptidase, which are converted in the small intestine to their active form trypsin, chymotrypsin, and carboxypeptidase, respectively.

Ongoing effort to improve the method for characterization of the complex proteins leads this invention to creates such a new and useful method. Given to the complexities of pancreatic protein, there is a need to apply robust technique to monitor the critical quality attributes of pancreatic protein mixture. The quality assessment is imperative to comply with regulatory agency guidelines, one of these attributes is a quantitative assessment of the aggregation, including dimers and multimers, low molecular weight, high molecular weight protein present in the pancreatic protein mixture. The presence of undesired protein or quantity of protein in pancreatic drug substance compromise the safety and efficacy of patient. Current method is an improved method which attempt to analyze the pancrelipase by using Size Exclusion High Performance Liquid Chromatography (SE-HPLC).

SE-HPLC is an important tool to determine the qualitative similarity between test sample (protein mixture) and reference product standard. In an absence of adequate method, it would not be possible to separate all proteins in protein mixture based on their size and thereby qualitative assessment cannot be carried out with reference product standard. The present invention provides a pharmaceutical acceptable pancrelipase protein.

### SUMMARY OF THE INVENTION

The invention provides a method for analysis of pancreatic protein mixture comprising an enzyme selected from amylase, protease, lipase and combination thereof.

In one aspect, the present invention provides a process to improve the profile of pancreatic protein mixture comprising at least one enzyme amylase, protease and lipase through SE-HPLC by reducing or controlling at least one undesired impurity.

In another aspect, the present invention provides a process to improve batch to batch consistency to comply with regulatory guideline.

The present invention provides an improved method for analysis of pancreatic protein mixture comprises at least more than one biological active protein, wherein the analysis of protein mixtures is performed with Size Exclusion High Performance Liquid Chromatography (SE-HPLC).

The present invention provides an improved method for quantification of pancreatic protein mixture comprises at least more than one biological active protein, wherein the quantification of protein mixtures is performed with Size Exclusion High Performance Liquid Chromatography (SE-HPLC).

The present invention provides an improved method for separation or quantification of the low molecular weight and high molecular weight pancreatic enzymes. In certain embodiment, the method provides the separation or quantification of HMW of enzymes selected from amylase, protease and lipase. In certain embodiment, the method provides the separation or quantification of LMW of enzymes selected from amylase, protease and lipase.

The present invention provides an improved method for analysis of the low molecular weight and high molecular weight pancreatic enzymes.

The present invention provides an improved method for quantification of the low molecular weight and high molecular weight pancreatic enzymes.

In an embodiment, the present invention provides an improved method for the size-based separation of pancrelipase enzymes comprising the mixture of at least amylase, lipase and protease by using Size Exclusion High Performance Liquid Chromatography (SE-HPLC), wherein the SE-HPLC is performed with organic solvent or/and suitable buffer.

In an embodiment, the present invention provides the method for separating and analyzing of pancreatic enzymes present in pancreatic protein mixture comprising:
a. preparing the soluble protein mixture from pancreatic sample;
b. loading the soluble protein mixture onto SE-HPLC column;
c. treating the SE-HPLC column with suitable separating solution in mobile phase selected from buffer or/and organic solvent;
d. eluting the pancreatic enzyme based on their molecular weight;
e. analyzing the eluted pancreatic enzyme comprising at least one enzyme selected from amylase, protease, and lipase.

In an embodiment, the present invention provides a method for the quantification of pancreatic enzymes present in pancreatic protein mixture comprising:
a. preparing the soluble protein mixture from pancreatic sample;
b. loading the protein mixture onto SE-HPLC column;
c. treating the SE-HPLC column with suitable separating solution in mobile phase selected from buffer or/and organic solvent;
d. eluting the pancreatic enzyme based on their molecular weight;
e. quantifying the eluted pancreatic enzyme comprising at least one enzyme selected from amylase, protease, and lipase.

In certain embodiment, the pancreatic protein mixture is obtained from crude, partially purified and substantially purified and microbially synthesize pancreatic protein sample.

In certain embodiment, the suitable organic solvent is selected from iso-propyl alcohol (IPA), acetonitrile (ACN), methanol, trifluoro acetic acid (TFA), formic acid, and mixture thereof.

In certain embodiment, the suitable buffer is selected from citrate buffer, phosphate buffer, bicarbonate buffer or mixtures thereof.

In one embodiment, the present invention provides the method for separating and analyzing of pancreatic enzymes present in pancreatic protein mixture comprising:
a. preparing the soluble protein mixture from pancreatic sample;
b. treated the protein mixture with suitable reducing agent;
c. loading the soluble protein mixture onto SE-HPLC column;
d. treating the SE-HPLC column with suitable separating solution in mobile phase selected from buffer, or/and organic solvent;
e. eluting the pancreatic enzyme based on their molecular weight;
f. analyzing the eluted pancreatic enzyme comprising at least one enzyme selected from amylase, protease, and lipase.

In one embodiment, the present invention provides the quantification of pancreatic enzymes present in pancreatic protein mixture comprising:
a. preparing the soluble protein mixture from pancreatic sample;
b. treated the protein mixture with suitable reducing agent loading the protein mixture onto SE-HPLC column;
c. treating the SE-HPLC column with suitable separating solution in mobile phase selected from buffer or/and organic solvent;
d. eluting the pancreatic enzyme based on their molecular weight;
e. f quantifying the eluted pancreatic enzyme comprising at least one enzyme selected from amylase, protease, and lipase

The suitable reducing agent are selected from dithiothreitol (DTT), β-mercaptoethanol (β-MCE), and tris(2-carboxyethyl)phosphine (TCEP).

### DETAILED DESCRIPTION OF FIGURE

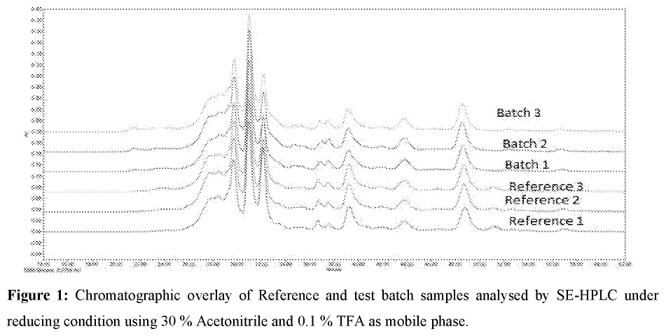

FIG. 1: shows, a chromatographic overlay of reference and test batch samples analysed by SE-HPLC under reducing condition using 30% acetonitrile and 0.1% TFA as mobile phase.

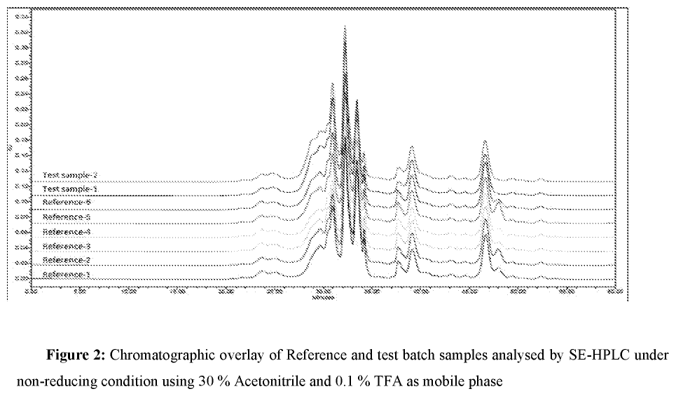

FIG. 2: shows, a chromatographic overlay of reference and test batch samples analysed by SE-HPLC under non-reducing condition using 30% acetonitrile and 0.1% TFA as mobile phase

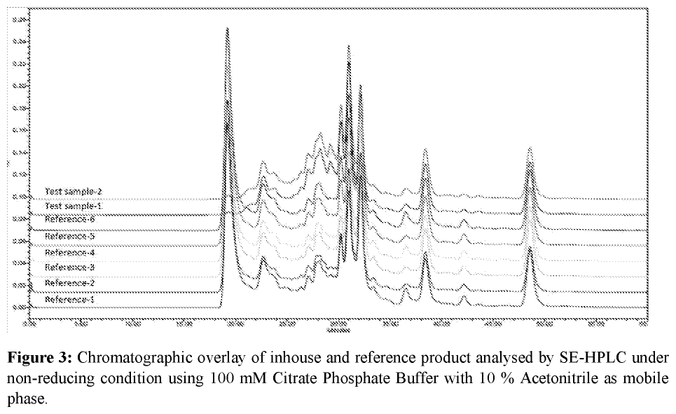

FIG. 3: shows, a chromatographic overlay of inhouse and reference product analysed by SE-HPLC under non-reducing condition using 100 mM citrate phosphate buffer with 10% acetonitrile as mobile phase.

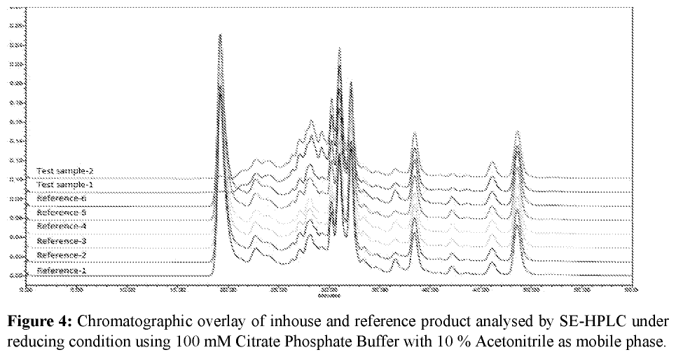

FIG. 4: shows, a chromatographic overlay of inhouse and reference product analysed by SE-HPLC under reducing condition using 100 mM citrate phosphate buffer with 10% acetonitrile as mobile phase.

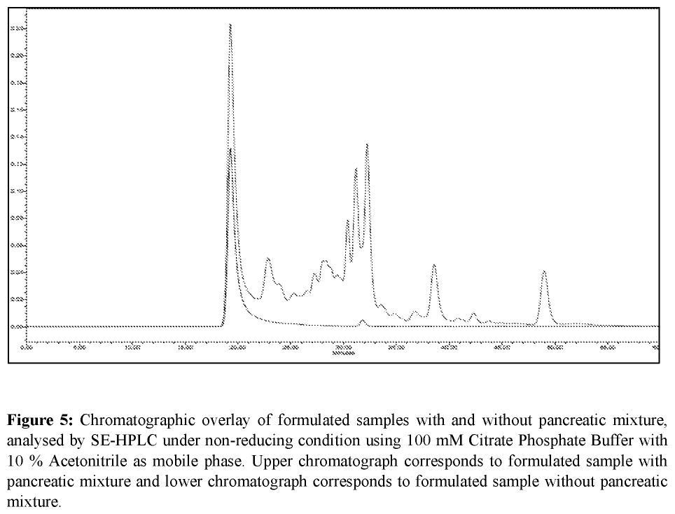

FIG. 5: shows, a chromatographic overlay of formulated samples with and without pancreatic mixture, analysed by SE-HPLC under non-reducing condition using 100 mM citrate phosphate buffer with 10% acetonitrile as mobile phase. Upper chromatograph corresponds to formulated sample with pancreatic mixture and lower chromatograph corresponds to formulated sample without pancreatic mixture.

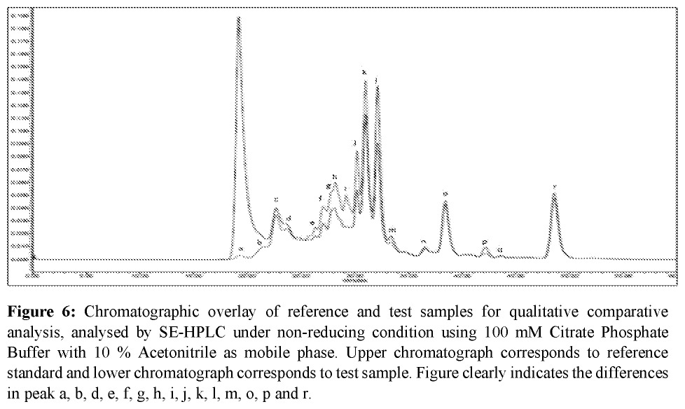

FIG. 6: shows chromatographic overlay of reference and test samples for qualitative comparative analysis, analysed by SE-HPLC under non-reducing condition using 100 mM citrate phosphate buffer with 10% acetonitrile as mobile phase. Upper chromatograph corresponds to reference standard and lower chromatograph corresponds to test sample. Figure clearly indicates the differences in peak a, b, d, e, f, g, h, i, j, k, l, m, o, p, and r.

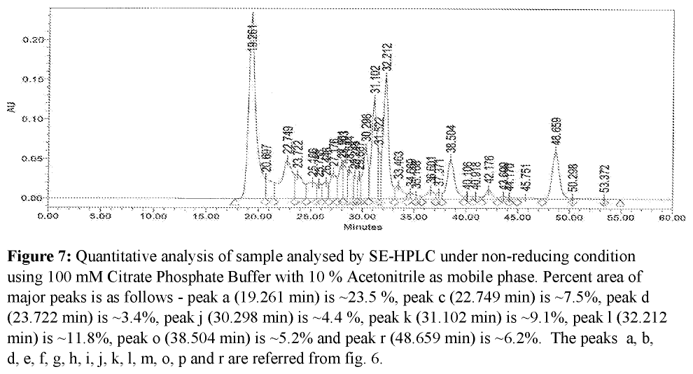

FIG. 7: shows, a quantitative analysis of sample analysed by SE-HPLC under non-reducing condition using 100 mM Citrate Phosphate Buffer with 10% Acetonitrile as mobile phase. Percent area of major peaks is as follows—peak a (19.261 min) is ˜23.5%, peak c (22.749 min) is ˜7.5%, peak d (23.722 min) is ˜3.4%, peak j (30.298 min) is ˜4.4%, peak k (31.102 min) is ˜9.1%, peak l (32.212 min) is ˜11.8%, peak o (38.504 min) is ˜5.2% and peak r (48.659 min) is ˜6.2%. The peaks a, b, d, e, f, g, h, i, j, k, l, m, o, p and r are referred from FIG. 6.

### DETAILED DESCRIPTION OF THE EMBODIMENTS

### Definitions

Unless the context clearly requires otherwise, throughout the invention, the words “comprise”, “comprising”, and the like are to be construed in an inclusive sense as opposed to an exclusive or exhaustive sense; that is to say, in the sense of “including, but not limited to”.

The term “about” as used herein is intended to refer to ranges of approximately 10% to 20% greater than or less than the referenced value. In certain circumstances, one skill in the art will recognize that, due to the nature of the referenced value, the term about can mean more or less than a 10% to 20% deviation from that value.

The term “pancrelipase samples” or “pancreatic sample” or “pancreatic protein sample” refers to pancreatic digestive enzymes formulated in any pharmaceutical composition. In an embodiment, the pancrelipase sample is selected from granules, tablet, capsules and powder. The “pancrelipase samples” or “pancreatic sample” or “pancreatic protein sample” comprises at one enzyme selected from lipase, protease, amylase and combination thereof. In an embodiment, the “pancrelipase samples” or “pancreatic sample” or “pancreatic protein sample” obtained from crude, partially purified, substantially purified and microbially synthesize.

The term “separating solution” refers to suitable buffer, suitable organic solvent and combination thereof used in mobile phase of SE-HPLC to separate the pancreatic enzymes present in pancreatic protein mixture based on their size

The term “protein variant” refers to a member of a set of highly similar or identical proteins that originate from a single gene or gene family and are the result of genetic differences. In an embodiment, the protein variant is at least 70% or about 75% or about 80% or about 85% or about 90% or about 91% or about 92% or about 93% or about 94% or about 95% or about 96% or about 97% or about 98% and about 99% identical or similar to protein of interest.

The term “HMW species” or “HMW” or “high molecular weight” refers to any one or more proteins with a higher apparent molecular weight relative to the intact protein of interest. The HMW species can be unrelated to the protein of interest or are aggregates, e.g., a dimer or multimer or any combination of the intact protein and any fragment thereof. These HMW species are pancreatic product-related variants that contribute to the size heterogeneity of protein products. In certain embodiment, the method provides the separation and/or quantification of HMW of enzymes selected from amylase, protease and lipase. The formation of HMW species within a drug product as a result of protein aggregation can potentially compromise both drug efficacy and safety.

The term “LMW species” or “LMW” or “low molecular weight” refers to one or more proteins with a lower apparent molecular weight relative to the intact protein of interest. The LMW species can be unrelated to the protein of interest or can be protein fragments. These LMW species which is a protein backbone-truncated fragments in pancreatic product that contribute to the size heterogeneity of protein. In certain embodiment the method provides the separation and/or quantification of LMW of enzymes selected from amylase, protease and lipase.

LMW species are considered critical quality attributes that are routinely monitored during drug development and as part of release testing of purified drug product during manufacturing.

In an embodiment, the pancreatic sample comprises enzyme selected from Triacylglycerol lipase, Co-lipase, CEL lipase, Phospholipase A2, Trypsin, Chymotrypsin, Elastase, Carboxypeptidase A1, Carboxypeptidase B, Kallikrien glandular, and Alpha amylase are the prominent functionally important enzymes.

The term “organic solvent” refers to solvent selected from IPA, acetonitrile (ACN), methanol, trifluoro acetic acid (TFA), formic acid, and mixtures thereof. In certain embodiment, the organic solvent is used in combination with pure water. In another embodiment, the organic solvent is used in combination with salt base or ionic solution. Organic solvent(s) is used for the preparation of suitable separating solution which is used in mobile phase of SE-HPLC.

The term “suitable buffer” refers to buffer selected from citrate buffer, phosphate buffer, citrate-phosphate buffer, bicarbonate. In certain embodiment, the buffer is used for the preparation of suitable separating solution which is used in mobile phase of SE-HPLC.

The term “reference standard” refers pancrelipase product which is approved by regulatory agencies FDA and EMA. In certain embodiment, the reference standard is selected from creon, Pancreaze, Pancrelipase, Pangestytne EC, Pangestyme C, Panocaps, Pertzye, Ultracaps, Ultresa, Viokace, Zenpep.

The term “test sample” refers to protein extract of test sample which is developed by the present applicant.

The term “mobile phase” is a solvent which helps to carry the mixture down in the column. In one aspect, the mobile phase comprises suitable organic solvent and/or suitable buffer or combination thereof.

The term “and/or” refers to one option could be “organic solvent and buffer” and another option could be “organic solvent or buffer.”

The term “qualitative comparison” refers to visual comparative analysis between reference sample and test sample by means of overlay.

The term “Size Exclusion High Performance Liquid Chromatography” or “SE-HPLC” refers to chromatography column itself contains fine and porous beads, which are composed of dextran, agarose, silica or polyacrylamide (the stationary phase). The beads allow small species to migrate into their pores, increasing their retention inside the column, while larger species migrate with the mobile phase without entering the bead pores. Thus, the larger species reach the column end faster than smaller species. After separation, the various species are detected using following detectors: Ultraviolet (UV) absorption, Fluorescence, Refractive Index (RI), Multi-angle Laser Light Scattering (MALLS), Charged Aerosol Detection (CAD). The skilled person is able to select any SE-HPLC column based on their interest and in view of the principle described above.

SE-HPLC allows the sizing, quantification and molecular weight determination of fragments, monomers and aggregates. The size range of HP-SEC is defined by the pore size of the column and the method set-up (e.g., mobile phase, flow settings and column dimensions).

In an embodiment, the invention provides a pharmaceutically acceptable pancreatic protein mixture comprising one or more enzymes selected from amylase, lipase and protease.

In one embodiment, the analysis is performed by method selected from CE-SDS, SDS-PAGE, MALDI-TOF-MS, MS, RP-HPLC, RP-UHPLC and visual observation of SE-HPLC profile.

In one embodiment, the invention provides a method for quantification and/or analysis of pancreatic protein mixture comprising at least one enzyme amylase, protease and lipase through SE-HPLC.

In one embodiment, the quantification is performed by method selected from CE-SDS, SDS-PAGE, MALDI-TOF-MS, MS, RP-HPLC, RP-UHPLC and SE-HPLC.

In one embodiment, the invention provides a method for analysis of pancreatic protein mixture comprising an enzyme selected from amylase, protease, lipase and combination thereof.

In one embodiment, the invention provides a method for analysis of pancreatic protein mixture comprising a low molecular weight and high molecular weight pancreatic enzymes selected from amylase, protease, lipase and combination thereof. In certain embodiment, the method provides the separation of HMW of enzymes selected from amylase, protease and lipase. In certain embodiment, the method provides the separation of LMW of enzymes selected from amylase, protease and lipase.

In one embodiment, the invention provides a method for quantification of pancreatic protein mixture comprising an enzyme selected from amylase, protease, lipase and combination thereof.

In one embodiment, the invention provides a method for quantification provides quantification of pancreatic protein mixture comprising a low molecular weight and high molecular weight pancreatic enzymes selected from amylase, protease, lipase and combination thereof. In certain embodiment, the method provides the quantification of HMW of enzymes selected from amylase, protease and lipase. In certain embodiment the method provides the quantification of LMW of enzymes selected from amylase, protease and lipase.

In one embodiment, the present invention provides a process to improve the profile of pancreatic protein mixture comprising at least one enzyme amylase, protease and lipase through SE-HPLC by reducing or controlling at least one undesired impurity.

In another embodiment, the present invention provides a process to improve batch to batch consistency to comply with regulatory guideline.

In one embodiment, the present invention provides an improved method for analysis of pancreatic protein mixture comprises at least more than one biological active protein, wherein the analysis of protein mixtures is performed with Size Exclusion High Performance Liquid Chromatography (SE-HPLC).

In one embodiment, the present invention provides an improved method for quantification of pancreatic protein mixture comprises at least more than one biological active protein, wherein the quantification of protein mixtures is performed with Size Exclusion High Performance Liquid Chromatography (SE-HPLC).

In one embodiment, the present invention provides an improved method for quantification of the low molecular weight and high molecular weight pancreatic enzymes. In certain embodiment, the method provides the quantification of HMW of enzymes selected from amylase, protease and lipase. In certain embodiment, the method provides the quantification of LMW of enzymes selected from amylase, protease and lipase.

In an embodiment, the present invention provides an improved method for the size-based separation of pancrelipase enzymes comprising the mixture of at least amylase, lipase and protease by using Size Exclusion High Performance Liquid Chromatography (SE-HPLC), wherein the SE-HPLC is performed with organic solvent or/and suitable buffer.

In an embodiment, the SE-HPLC provides 18 to 25 major protein peaks.

In another embodiment, the SE-HPLC provides 18 major protein peaks.

In another embodiment, the major protein peaks are selected from PLA2, Triacylglycerol lipase (TAG) lipase, Colipase, Trypsin, Elastase, Chymotrypsin, Carboxypeptidase-A (CPA), Carboxypeptidase-B (CPB), Amylase and variant thereof.

In an embodiment, the method provides the quantification of HMW of amylase. In an embodiment, the method provides the quantification of LMW of amylase.

In an embodiment, the method provides the quantification of HMW of lipase. In an embodiment, the method provides the quantification of LMW of lipase.

In an embodiment, the method provides the quantification of HMW of protease. In an embodiment, the method provides the quantification of LMW of protease.

In an embodiment, the method provides the analysis of HMW of amylase. In an embodiment, the method provides the analysis of LMW of amylase.

In an embodiment, the method provides the analysis of HMW of lipase. In an embodiment, the method provides the analysis of LMW of lipase.

In an embodiment, the method provides the analysis of HMW of protease. In an embodiment, the method provides the analysis of LMW of protease.

In an embodiment, the present invention provides the method for separating and analyzing of pancreatic enzymes present in pancreatic protein mixture comprising:
a. preparing the soluble protein mixture from pancreatic sample;
b. loading the soluble protein mixture onto SE-HPLC column;
c. treating the SE-HPLC column with suitable separating solution in mobile phase selected from buffer or/and organic solvent;
d. eluting the pancreatic enzyme based on their molecular weight;
e. analyzing the eluted pancreatic enzyme comprising at least one enzyme selected from amylase, protease, and lipase.

In an embodiment, the present invention provides a method for the quantification of pancreatic enzymes present in pancreatic protein mixture comprising:
a. preparing the soluble protein mixture from pancreatic sample;
b. loading the protein mixture onto SE-HPLC column;
c. treating the SE-HPLC column with suitable separating solution in mobile phase selected from buffer or/and organic solvent;
d. eluting the pancreatic enzyme based on their molecular weight;
e. quantifying the eluted pancreatic enzyme comprising at least one enzyme selected from amylase, protease, and lipase.

In certain embodiment, the pancreatic protein mixture is obtained from crude, partially purified, substantially purified and microbially synthesize pancreatic protein sample.

In one embodiment, the suitable organic solvent is selected from iso-propyl alcohol (IPA), acetonitrile (ACN), methanol, trifluoro acetic acid (TFA), formic acid, and mixture thereof.

In certain embodiment, the suitable organic solvent has concentration selected from about 5% to about 40% in mobile phase. In certain embodiment, the suitable organic solvent has concentration selected from about 5% to about 30% in mobile phase. In certain embodiment, the suitable organic solvent has concentration selected from about 5% to about 20% in mobile phase. In an embodiment, the suitable organic solvent has concentration selected from about 5% to about 10% in mobile phase.

In an embodiment, the suitable organic solvent has concentration selected from about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, and about 40% in mobile phase.

In an embodiment, the mobile phase comprises organic solvent in combination with aqueous solution.

In certain embodiment, the organic solvent acetonitrile (ACN) has concentration about 5% to about 40% in mobile phase.

In certain embodiment, the acetonitrile (ACN) has concentration selected from about 5% to about 40% in mobile phase. In certain embodiment, the acetonitrile (ACN) has concentration selected from about 5% to about 30% in mobile phase. In certain embodiment, the acetonitrile (ACN) has concentration selected from about 5% to about 20% in mobile phase. In an embodiment, the acetonitrile (ACN) has concentration is selected from about 5% to about 10% in mobile phase. In an embodiment, the organic solvent comprises about 10% acetonitrile (ACN) in mobile phase.

In an embodiment, the organic solvent acetonitrile (ACN) has concentration selected about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 32%, about 33%, about 34%, about 35%, about 36%, about 37%, about 38%, about 39%, about 40% in mobile phase.

In an embodiment, the organic solvent is iso-propyl alcohol (IPA) has concentration about 5% to about 40% in mobile phase.

In an embodiment, the organic solvent is methanol has concentration about 5% to about 40% in mobile phase.

In an embodiment, the mobile phase comprises of acetonitrile (ACN) having concentration about 5% to about 40%, and formic acid having concentration about 0.05% to about 0.3%.

In an embodiment, the mobile phase comprises of acetonitrile (ACN) having concentration about 5% to about 40% and trifluoro acetic acid (TFA) having concentration about 0.05% to about 0.3%.

In an embodiment, the mobile phase comprises of acetonitrile (ACN) having concentration about 30% and trifluoro acetic acid (TFA) having concentration about 0.1%.

In an embodiment, the mobile phase comprises combination of acetonitrile (ACN) concentration about 30%, trifluoro acetic acid (TFA) having concentration about 0.1%, and aqueous solution.

In one embodiment, the suitable buffer used in separation is selected from citrate buffer, phosphate buffer, bicarbonate buffer or mixtures thereof.

In one embodiment, the suitable buffer is citrate-phosphate buffer.

In one embodiment, the concentration of buffer is selected from about 10 mM to about 200 mM.

In one embodiment, the concentration of buffer is selected from about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, about 200 mM.

In one embodiment, the concentration of citrate-phosphate buffer is selected from about 10 mM to about 200 mM.

In one embodiment, the concentration of citrate-phosphate buffer is selected from about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, about 200 mM.

In one embodiment, the concentration of citrate-phosphate buffer is selected from about 100 mM.

In one embodiment, the concentration of bicarbonate buffer is selected from about 10 mM to about 200 mM.

In one embodiment, the concentration of bicarbonate buffer is selected from about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, about 200 mM.

In one embodiment, the concentration of citrate buffer is selected from about 10 mM to about 200 mM.

In one embodiment, the concentration of citrate buffer is selected from about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, about 200 mM.

In one embodiment, the concentration of phosphate buffer is selected from about 10 mM to about 200 mM.

In one embodiment, the concentration of phosphate buffer is selected from about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, about 200 mM.

In one embodiment, the pH of the buffer is selected from about 5 to about 6.7.

In one embodiment, the pH of the buffer is selected from about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7.

In one embodiment, the pH of the buffer is 6.20.

In one embodiment, the buffer solution prepares in suitable solvent. In other embodiment, the suitable solvent for buffer preparation is selected from organic solvent and water.

In one embodiment, the suitable organic solvent is selected from iso-propyl alcohol (IPA), acetonitrile (ACN), methanol, trifluoro acetic acid (TFA), formic acid, and mixture thereof having concentration of about 5% to about 40% in combination with the suitable buffer is selected from citrate buffer, phosphate buffer, bicarbonate buffer, citrate phosphate buffer and mixture thereof having concentration selected from about 10 mM to about 200 mM.

In one embodiment, the suitable organic solvent having concentration of about 5% to about 40% in combination with suitable buffer having concentration selected from about 10 mM to about 200 mM.

In one embodiment, the suitable organic solvent having concentration of about 5% to about 40% in combination with citrate-phosphate buffer having concentration selected from about 10 mM to about 200 mM.

In one embodiment, the organic solvent is acetonitrile (ACN) having concentration of about 5% to about 40% in combination with citrate-phosphate buffer having concentration selected from about 10 mM to about 200 mM.

In one embodiment, the organic solvent is acetonitrile (ACN) having concentration of about 5% in combination with about 100 mM citrate phosphate buffer.

In one embodiment, the organic solvent is acetonitrile (ACN) having concentration of about 10% in combination with 100 mM citrate phosphate buffer.

In one embodiment, the organic solvent acetonitrile (ACN) having concentration of about 20% in combination with the 100 mM citrate phosphate buffer.

In one embodiment, the organic solvent is acetonitrile (ACN) having concentration of about 30% in combination with 100 mM citrate phosphate buffer.

In one embodiment, the organic solvent is acetonitrile (ACN) having concentration of about 40% in combination with 100 mM citrate phosphate buffer.

In one embodiment, the organic solvent is iso-propyl alcohol (IPA) having concentration of about 5% to about 40% in combination with citrate-phosphate buffer having concentration selected from about 10 mM to about 200 mM.

In one embodiment, the organic solvent is methanol having concentration of about 5% to about 40% in combination with citrate-phosphate buffer having concentration selected from about 10 mM to about 200 mM.

In one embodiment, the organic solvent having concentration of about 5% to about 40% in combination with citrate buffer having concentration selected from about 10 mM to about 200 mM.

In one embodiment, the organic solvent is acetonitrile (ACN) having concentration of about 5% to about 40% in combination with citrate buffer having concentration selected from about 10 mM to about 200 mM.

In one embodiment, the organic solvent is iso-propyl alcohol (IPA) having concentration of about 5% to about 40% in combination with citrate buffer having concentration selected from about 10 mM to about 200 mM.

In one embodiment, the organic solvent is methanol having concentration of about 5% to about 40% in combination with citrate buffer having concentration selected from about 10 mM to about 200 mM.

In one embodiment, the organic solvent is having concentration of about 5% to about 40% in combination with phosphate buffer having concentration selected from about 10 mM to about 200 mM.

In one embodiment, the organic solvent is acetonitrile (ACN) having concentration of about 5% to about 40% in combination with phosphate buffer having concentration selected from about 10 mM to about 200 mM.

In one embodiment, the organic solvent is iso-propyl alcohol (IPA) having concentration of about 5% to about 40% in combination with phosphate buffer having concentration selected from about 10 mM to about 200 mM.

In one embodiment, the organic solvent is methanol having concentration of about 5% to about 40% in combination with phosphate buffer having concentration selected from about 10 mM to about 200 mM.

In one embodiment, the suitable organic solvent is selected from iso-propyl alcohol (IPA), acetonitrile (ACN), methanol, trifluoro acetic acid (TFA), formic acid, and mixture having concentration of about 5% to about 40% in combination with bicarbonate buffer having concentration selected from about 10 mM to about 200 mM.

In one embodiment, the organic solvent is having concentration of about 5% to about 40% in combination with bicarbonate buffer having concentration selected from about 10 mM to about 200 mM.

In one embodiment, the organic solvent is acetonitrile (ACN) having concentration of about 5% to about 40% in combination with bicarbonate buffer having concentration selected from about 10 mM to about 200 mM.

In one embodiment, the organic solvent is iso-propyl alcohol (IPA) having concentration of about 5% to about 40% in combination with bicarbonate buffer having concentration selected from about 10 mM to about 200 mM.

In one embodiment, the organic solvent is methanol having concentration of about 5% to about 40% in combination with bicarbonate buffer having concentration selected from about 10 mM to about 200 mM.

In one embodiment, the organic solvent is acetonitrile (ACN) having concentration of about 30% with about 0.1% trifluoro acetic acid (TFA) in combination with buffer having concentration selected from about 10 mM to about 200 mM.

In one embodiment, the organic solvent is acetonitrile (ACN) having concentration of about 30% with about 0.1% trifluoro acetic acid (TFA) in combination with citrate-phosphate buffer having concentration selected from about 10 mM to about 200 mM.

In another embodiment, the pancreatic protein mixture is treated with suitable reducing agent before loading onto SE-HPLC column.

In one embodiment, the present invention provides the method for separating and analyzing of pancreatic enzymes present in pancreatic protein mixture comprising:
a. preparing the soluble protein mixture from pancreatic sample;
b. treated the protein mixture with suitable reducing agent;
c. loading the soluble protein mixture onto SE-HPLC column;
d. treating the SE-HPLC column with suitable separating solution in mobile phase selected from buffer, or/and organic solvent;
e. eluting the pancreatic enzyme based on their molecular weight;
f. analyzing the eluted pancreatic enzyme comprising at least one enzyme selected from amylase, protease, and lipase.

In an embodiment, the present invention provides a method for the quantification of pancreatic enzymes present in pancreatic protein mixture comprising:
a. preparing the soluble protein mixture from pancreatic sample;
b. loading the protein mixture onto SE-HPLC column;
c. treating the SE-HPLC column with suitable separating solution in mobile phase selected from buffer or/and organic solvent;
d. eluting the pancreatic enzyme based on their molecular weight;
e. quantifying the eluted pancreatic enzyme comprising at least one enzyme selected from amylase, protease, and lipase.

In one embodiment, the suitable reducing agent are selected from dithiothreitol (DTT), β-mercaptoethanol (β-MCE), and tris(2-carboxyethyl)phosphine (TCEP).

In an embodiment, the 1M DTT (Dithiothreitol) solution as reducing agent is used to reduce the reference sample and the test sample.

In an embodiment, the final concentration of DTT in sample is 10 mM.

In an embodiment, the reduction process of reference/test sample, 4 μl of 1M DTT solution is added and mixed into 396 μl reference standard/test samples.

In an embodiment, the reduced sample is incubated at 37° C. for 30 minutes in dry bath.

In an embodiment, the HPLC system equipped with a pump, an autosampler, a UV detector and a suitable data acquisition system.

In an embodiment, the column is selected from Biobasic SEC 120 column.

In an embodiment, the detection is carried out at UV frequency 280 nm.

In an embodiment, the elution is performed in isocratic gradient.

In an embodiment, the elution is performed in isocratic mode.

In an embodiment, the flow rate is maintained from about 0.1 ml/min, about 0.2 ml/min, about 0.3 ml/min, about 0.4 ml/min, about 0.5 ml/min, about 0.6 ml/min, ml/min, about 0.7 ml/min, about 0.8 ml/min, about 0.9 ml/min and about 1 ml/min.

In certain embodiment, the flow rate is maintained at about 0.3 ml/min.

In an embodiment, the injection amount of a pancreatic sample is selected from about 10 μg, about 20 μg, about 30 μg, about 40 μg, about 50 μg.

In an embodiment, the injection amount of a pancreatic sample is about 40 μg.

In an embodiment, the column temperature is maintained from about 25° C., about 26° C., about 27° C., about 28° C., about 29° C., about 30° C.

In an embodiment, the column temperature is maintained at about 30° C.

In an embodiment, the sample temperature is maintained from about 5° C., about 6° C., about 7° C., about 8° C., about 9° C., about 10° C.

In an embodiment, the sample temperature is maintained at about 6° C.

In an embodiment, the sample run for about 30 minutes, about 40 minutes, about 50 minutes, about 60 minutes, about 70 minutes, about 80 minutes, about 90 minutes.

In an embodiment, the sample run for about 60 minutes.

In an embodiment, the needle is washed with 5% (v/v) methanol in water.

### EXAMPLES

The present invention provides below examples for illustrative purpose only and invention should not be considered limiting to below examples.

### Example 1

Equipment used in the present invention are given below:

HPLC equipped with a pump, an autosampler, a UV detector and a suitable data acquisition system, Biobasic SEC 120 Column, (7.8 mm ID, Length 300 mm, 5 μm), Adjustable volume pipettes and tips, Sonicator and, Standard laboratory cleaned glassware.

Equipment used in the present invention are given below:

HPLC equipped with a pump, an autosampler, a UV detector and a suitable data acquisition system, Biobasic SEC 120 Column, (7.8 mm ID, Length 300 mm, 5 μm), Adjustable volume pipettes and tips, Sonicator and, Standard laboratory cleaned glassware.

Procedure: Prepared 1M of DTT (Dithiothreitol) solution for the reduction of reference/test sample, to prepare 1M of DTT solution, 1.54.3 mg of DTT dissolved in 1 ml of milli Q water. Further, a reference standard and the test sample were reduced with DTT, wherein the final concentration of DTT in sample was 10 mM. For this reduction process, added 4 μl of 1M DTT solution into 396 μl reference standard/test samples. Mixed it well. Incubated the samples at 37° C. for 30 minutes in dry bath. From this reduced sample injected 40 μg protein amount by varying injection volume based on measured protein concentration for further analysis. The separation was achieved by using Biobasic SEC 120 column with isocratic elution using an organic phase as a mobile phase with 30% ACN with 0.1% TFA and detected by UV at 280 nm. Flow rate was maintained at 0.3 ml/min. The column temperature maintained at 30° C. and the sample temperature kept at 6° C. Sample run in the system for 60 minutes. Needle washed with 5% (v/v) methanol in water.

Results: Qualitative comparative analysis between reference standard and test samples is shown by showing an overlay of reference standard with test samples. It is evident from FIG. 1 that test samples are similar to reference standard.

### Example 2

For non-reduce analysis, test sample and reference standard were injected with 40 μg of protein amount.

The separation was achieved by using Biobasic SEC 120 column with isocratic elution using an organic phase as a mobile phase with 30% ACN with 0.1% TFA and detected by UV at 280 nm. Flow rate was maintained at 0.3 ml/min. The column temperature maintained at 30° C. and the sample temperature kept at 6° C. Sample run in the system for 60 minutes. Needle washed with 5% (v/v) methanol in water.

Results: Qualitative comparative analysis between reference standard and test samples is shown by showing an overlay of reference standard with test samples. It is evident from FIG. 2 that test samples are similar to reference standard.

### Example 3

For non-reduce analysis, test sample and reference standard were injected with 40 μg of protein amount.

The separation achieved by using Biobasic SEC 120 column (7.8 mm ID, Length 300 mm, 5 μm) with isocratic elution using 100 mM citrate phosphate buffer with 10% Acetonitrile and detected by UV at 280 nm. Flow rate was maintained at 0.3 mL/min. The column temperature maintained at 30° C. and sample temperature kept at 6° C. Sample run in the system 60 minutes. Needle washed with 5% (v/v) methanol in water. For the obtained results refer FIG. 3.

It is evident from FIG. 3 that qualitative overlay is consistent across batches of pancreatic protein mixture and further shows the robustness of this method to identify differences among protein mixtures.

FIG. 5 shows the specificity of this method to identify and differentiate formulated sample with pancreatic mixture and without pancreatic mixture.

FIG. 6 further substantiate the ability of method to differentiate among protein mixtures samples in a peak specific manner by means of qualitative overlay. About 18 major peaks peak “a” to “r” are visually differentiated by qualitative analysis.

Quantitative analysis is shown in FIG. 7 for one of the pancreatic mixture samples to identify and quantitate each peak with respect to retention time and percent area.

### Example 4

For reducing analysis, test sample and reference standard were reduced with DTT, wherein the final concentration of DTT in sample was 10 mM. For this reduction process, added 4 μL of 1M DTT solution into 396 μL test sample (concentration 1 mg/mL). From this reduced sample injected 40 μg protein amount. The separation achieved by using Biobasic SEC 120 column (7.8 mm ID, Length 300 mm, 5 μm) with isocratic elution using 100 mM citrate phosphate buffer with 10% Acetonitrile and detected by UV at 280 nm. Flow rate was maintained at 0.3 mL/min. The column temperature maintained at 30° C. and sample temperature kept at 6° C. Sample run in the system 60 minutes. Needle washed with 5% (v/v) methanol in water. For the obtained results refer FIG. 4. As described in example 3, both qualitative and quantitative analysis can be performed with reduced pancreatic mixture samples.

1. A method for separating and analyzing pancreatic enzymes present in pharmaceutically acceptable pancreatic protein mixture comprising:
a. preparing the soluble protein mixture from pancreatic sample;
loading the soluble protein mixture onto SE-HPLC column;
treating the SE-HPLC column with suitable separating solution in mobile phase selected from buffer or/and organic solvent;
eluting the pancreatic enzyme based on their molecular weight;
analyzing the eluted pancreatic enzyme comprising at least one enzyme selected from amylase, protease, and lipase.

2. A method for the quantification of pancreatic enzymes present in pharmaceutically acceptable pancreatic protein mixture comprising;
preparing the soluble protein mixture from pancreatic sample;
loading the protein mixture onto SE-HPLC column;
treating the SE-HPLC column with suitable separating solution in mobile phase selected from buffer or/and organic solvent;
eluting the pancreatic enzyme based on their molecular weight;
quantifying the eluted pancreatic enzyme comprising at least one enzyme selected from amylase, protease, and lipase.

3. The method according to claim 1, or claim 2, wherein the pancreatic protein mixture is obtained from crude, partially purified, substantially purified and microbially synthesize pancreatic protein sample.

4. The method according to claim 1, wherein the method provides analysis of pancreatic protein mixture comprising an enzyme selected from amylase, protease, lipase and combination thereof.

5. The method according to claim 1, wherein the method provides analysis of pancreatic protein mixture comprising a low molecular weight and high molecular weight impurities of pancreatic enzymes selected from amylase, protease, lipase and combination thereof.

6. The method according to claim 1 or claim 2 wherein the analysis or quantification is performed by method selected from CE-SDS, SDS-PAGE, MALDI-TOF-MS, RP-HPLC, RP-UHPLC, MS and SE-HPLC.

7. The method according to claim 2 wherein the method provides quantification of pancreatic protein mixture comprising an enzyme selected from amylase, protease, lipase and combination thereof.

8. The method according to claim 2, wherein the method provides quantification of pancreatic protein mixture comprising a low molecular weight and high molecular weight impurities pancreatic enzymes selected from amylase, protease, lipase and combination thereof.

9. The method according to claim 1 or claim 2 wherein the SE-HPLC provides 18 to 25 major protein peaks.

10. The method according to claim 1 or claim 2 wherein the SE-HPLC provides 18 major protein peaks.

11. The method according to claim 10 wherein the major protein peaks are selected from PLA2, Triacylglycerol lipase (TAG) lipase, colipase, trypsin, elastase, chymotrypsin, carboxypeptidase-A (CPA), carboxypeptidase-B (CPB), amylase and variant thereof.

12. The method according to claim 1, or claim 2, wherein the method reduces or controls at least one undesired impurity.

13. The method according to claim 1, or claim 2, wherein the method improves batch to batch consistency.

14. The method according to claim 1, or claim 2, wherein the organic solvent selected from Iso-propyl alcohol (IPA), Acetonitrile (ACN), Methanol, Trifluoro acetic acid (TFA), Formic acid, and mixture thereof to form suitable separating solution in mobile phase.

15. The method according to claim 14, wherein the organic solvent concentration is selected from about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, and about 40% in mobile phase.

16. The method according to claim 14, wherein organic solvent is further used in combination with aqueous solution to form suitable separating solution in mobile phase.

17. The method according to claim 14, wherein the organic solvent is acetonitrile (ACN).

18. The method according to claim 14, wherein the organic solvent is iso-propyl alcohol (IPA).

19. The method according to claim 14, wherein the organic solvent is methanol.

20. The method according to claim 14, wherein the organic solvent comprises of acetonitrile (ACN) having concentration about 5% to about 40%, and formic acid having concentration about 0.05% to about 0.3%.

21. The method according to claim 14, wherein the organic solvent comprises of acetonitrile (ACN) having concentration about 5% to about 40% in combination with trifluoro acetic acid (TFA) having concentration about 0.05% to about 0.3%.

22. The method according to claim 14, wherein the organic solvent comprises of acetonitrile (ACN) having concentration about 30% in combination with trifluoro acetic acid (TFA) having concentration about 0.1%.

23. The method according to claim 14, wherein the organic solvent comprises combination of acetonitrile (ACN) having concentration about 30%, trifluoro acetic acid (TFA) having concentration about 0.1%, and aqueous solution.

24. The method according to claim 1 or claim 2, wherein the suitable buffer is selected from citrate buffer, phosphate buffer, citrate-phosphate buffer, bicarbonate buffer or mixture thereof.

25. The method according to claim 24, wherein the suitable buffer is citrate-phosphate buffer.

26. The method according to claim 24, wherein the suitable buffer concentration is selected from about 10 mM to about 200 mM.

27. The method according to claim 24, wherein the suitable buffer pH is selected from about 5 to about 6.7.

28. The method according to claim 27, wherein the pH is about 6.20.

29. The method according to claim 1 or claim 2, wherein the suitable organic solvent is selected from Iso-propyl alcohol (IPA), Acetonitrile (ACN), Methanol, Trifluoro acetic acid (TFA), Formic acid, and mixture thereof having concentration of about 5% to about 40% in combination with the suitable buffer is selected from citrate buffer, phosphate buffer, bicarbonate buffer, citrate phosphate buffer and mixture thereof having concentration selected from about 10 mM to about 200 mM.

30. The method according to claim 29, wherein the organic solvent concentration is about 5% to about 40% in combination with the citrate-phosphate buffer having concentration is selected from about 10 mM to about 200 mM.

31. The method according to claim 29, wherein the Acetonitrile (ACN) having concentration of about 5% to about 40% in combination with the citrate-phosphate buffer concentration selected from about 10 mM to about 200 mM.

32. The method according to claim 31, wherein the Acetonitrile (ACN) having concentration of about 10% in combination with the 100 mM citrate phosphate buffer.

33. The method according to claim 29, wherein the suitable organic solvent is selected from Iso-propyl alcohol (IPA), Acetonitrile (ACN), Methanol, Trifluoro acetic acid (TFA), Formic acid, and mixture having concentration of about 5% to about 40% in combination with bicarbonate buffer having concentration selected from about 10 mM to about 200 mM.

34. The method according to claim 29, wherein the Acetonitrile (ACN) concentration is about 30% with about 0.1% trifluoro acetic acid (TFA) in combination with buffer having concentration selected from about 10 mM to about 200 mM.

35. The method according to claim 29, wherein the Acetonitrile (ACN) concentration is about 30% with about 0.1% trifluoro acetic acid (TFA) and in combination with citrate-phosphate buffer having concentration selected from about 10 mM to about 200 mM.

36. The method for separating and analyzing of pancreatic enzymes present in pharmaceutically acceptable pancreatic protein mixture comprising;
a. preparing the soluble protein mixture from pancreatic sample;
b. treated the protein mixture with suable reducing agent;
c. loading the soluble protein mixture onto SE-HPLC column;
d. treating the SE-HPLC column with suitable separating solution in mobile phase selected from buffer, or/and organic solvent;
e. eluting the pancreatic enzyme based on their molecular weight;
f. analyzing the eluted pancreatic enzyme comprising at least one enzyme selected from amylase, protease, and lipase.

37. The method for the quantification of pancreatic enzymes present in pharmaceutically acceptable pancreatic protein mixture comprising;
a. preparing the soluble protein mixture from pancreatic sample;
b. treated the protein mixture with suable reducing agent;
c. loading the protein mixture onto SE-HPLC column;
d. treating the SE-HPLC column with suitable separating solution in mobile phase selected from buffer or/and organic solvent;
e. eluting the pancreatic enzyme based on their molecular weight;
f. quantifying the eluted pancreatic enzyme comprising at least one enzyme selected from amylase, protease, and lipase.

38. The method according to claim 36 or claim 37, wherein the suitable reducing agent are selected from dithiothreitol (DTT), β-mercaptoethanol (β-MCE), and tris(2-carboxyethyl)phosphine (TCEP).

39. The method according to claim 1 or claim 36, wherein the protein mixture is analysed at suitable UV frequency 280 nm.

40. The method according to claim 1 or claim 2 or claim 36 or claim 37, wherein the elution is performed in isocratic gradient.

41. The method according to claim 1 or claim 2 or claim 36 or claim 37, wherein the SE-HPLC column maintains flow rate selected from about 0.1 ml/min, about 0.2 ml/min, about 0.3 ml/min, about 0.4 ml/min, about 0.5 ml/min, about 0.6 ml/min, ml/min, about 0.7 ml/min, about 0.8 ml/min, about 0.9 ml/min and about 1 ml/min.

42. The method according to claim 1 or claim 2 or claim 36 or claim 37, wherein the pancreatic sample is injected in amount selected from about 10 μg, about 20 μg, about 30 μg, about 40 μg, about 50 μg.

43. The method according to claim 1 or claim 2 or claim 36 or claim 37, wherein the SE-HPLC column temperature is maintained from about 25° C., about 26° C., about 27° C., about 28° C., about 29° C., about 30° C.

44. The method according to claim 1 or claim 2 or claim 36 or claim 37, wherein the pancreatic sample temperature is maintained from about 5° C., about 6° C., about 7° C., about 8° C., about 9° C., about 10° C.

45. The method according to claim 1 or claim 2 wherein the analysis is performed by method selected from CE-SDS, SDS-PAGE, MALDI-TOF-MS, MS, RP-HPLC, RP-UHPLC and visual observation of SE-HPLC profile.

46. The method according to claim 1 or claim 2 or claim 36 or claim 37, wherein the method provides a pharmaceutically acceptable pancreatic protein mixture comprising one or more enzymes selected from amylase, lipase and protease.
